(19) United States
(12) Patent Application Publication
(10) Pub. No.: US 2025/0223637 A1
(43) Pub. Date: Jul. 10, 2025

(54) DEVICES INCLUDING OSMOTICALLY BALANCED BARRIERS, AND METHODS OF MAKING AND USING THE SAME

(71) Applicant: Illumina, Inc., San Diego, CA (US)

(72) Inventors: Xavier von Hatten, Cambridge (GB); Antonio Conde-Gonzalez, Cambridge (GB); Istvan Kocsis, Cambridge (GB); Alexandre Richez, Cambridge (GB)

(73) Assignee: Illumina, Inc., San Diego, CA (US)

(21) Appl. No.: 18/850,831

(22) PCT Filed: Mar. 29, 2023
(86) PCT No.: PCT/EP2023/058184

§ 371 Date: Sep. 25, 2024

Related U.S. Application Data
(60) Provisional application No. 63/325,723, filed on Mar. 31, 2022.

(51) Int. Cl. C12Q 1/6869; C12N 9/12; C12Q 1/48

(52) U.S. Cl. CPC C12Q 1/6869; C12N 9/1241; C12Q 1/48

(57) ABSTRACT

Devices including osmotically balanced barriers, and methods of making and using the same, are provided herein. A fluidic well may include a barrier having first and second sides. A first fluid within the fluidic well may contact the first side of the barrier, and may have a first composition including a first concentration of a salt. A second fluid within the fluidic well may contact the second side of the barrier and may have a second composition including a second concentration of the salt that is different than the first concentration. The difference between the first and second concentrations of the salt may generate a first osmotic pressure across the barrier. The second composition further may include a concentration of a compound other than the salt. The concentration of the compound may generate a second osmotic pressure across the barrier that opposes and substantially balances the first osmotic pressure.

### CROSS-REFERENCE TO RELATED APPLICATIONS

This application claims the benefit of U.S. Provisional Patent Application No. 63/325,723, filed Mar. 31, 2022 and entitled “DEVICES INCLUDING OSMOTICALLY BALANCED BARRIERS, AND METHODS OF MAKING AND USING THE SAME”, the entire contents of which are incorporated by reference herein.

### FIELD

This application relates to barriers between first and second fluids.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in a α-hemolysin nanopore. Or, for example, KF-DNA complexes have been distinguished on the basis of their properties when captured in an electric field atop an α-hemolysin nanopore. In still another example, polynucleotide sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a polynucleotide that is being synthesized. The charge blockade label is detected by the nanopore to determine the presence and identity of the incorporated nucleotide and thereby determine the sequence of a template polynucleotide. In still other examples, constructs include a transmembrane protein pore subunit and a nucleic acid handling enzyme.

However, such previously known devices, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved devices, systems, and methods for sequencing polynucleotides, which may include using membranes having nanopores disposed therein.

### SUMMARY

Devices including osmotically balanced barriers, and methods of making and using the same, are provided herein.

Some examples herein provide a device. The device may include a fluidic well including a barrier. The barrier may include a first side and a second side. The device may include a first fluid having a first composition within the fluidic well and in contact with the first side of the barrier. The first composition may include a first concentration of a salt. The device may include a second fluid having a second composition within the fluidic well and in contact with the second side of the barrier. The second composition may include a second concentration of the salt that is different than the first concentration of the salt. The difference between the first and second concentrations of the salt may generate a first osmotic pressure across the barrier. The second composition further may include a concentration of a compound other than the salt. The concentration of the compound may generate a second osmotic pressure across the barrier that opposes and substantially balances the first osmotic pressure.

In some examples, the first concentration of the salt is between about 1.1 and about 50 times the second concentration of the salt. In some examples, the first concentration of the salt is between about 1.5 and about 20 times the second concentration of the salt. In some examples, the first concentration of the salt is between about 2 and about 10 times the second concentration of the salt. In some examples, the first concentration of the salt is above about 150 mM, and the second concentration of the salt is below about 100 mM. In some examples, the first concentration of the salt is above about 250 mM, and the second concentration of the salt is below about 100 mM. In some examples, the first composition substantially does not include the compound.

In some examples, the concentration of the compound is between about 1.1 and about 50 times the first concentration of the salt. In some examples, the concentration of the compound is between about 1.5 and about 20 times the first concentration of the salt. In some examples, the concentration of the compound is between about 2 and about 10 times the first concentration of the salt. In some examples, the concentration of the compound is above about 100 mM.

In some examples, the compound is charge neutral. In some examples, the compound increases viscosity of the second fluid. In some examples, the compound includes an alcohol. In some examples, the compound includes a protein. In some examples, the compound includes a polysaccharide. In some examples, the polysaccharide includes trehalose or a cyclodextrin.

In some examples, the salt includes potassium chloride (KCl). In some examples, the first composition includes a first concentration of an aqueous buffer. In some examples, the second composition includes a second concentration of the aqueous buffer. In some examples, the first concentration of the aqueous buffer is approximately equal to the second concentration of the aqueous buffer.

In some examples, the device further includes a nanopore disposed within the barrier and providing an aperture fluidically coupling the first side to the second side. In some examples, a portion of the salt moves from the second side of the barrier to the first side of the barrier through the aperture. In some examples, the compound substantially does not move from the second side of the barrier to the first side of the barrier through the aperture. In some examples, the compound is larger in at least one dimension than the aperture. In some examples, the device further includes a polymerase in the second composition or coupled to the nanopore or the barrier. In some examples, the compound stabilizes the polymerase. In some examples, the compound includes a co-factor of the polymerase. In some examples, the device further includes first and second polynucleotides. In some examples, the second composition further includes a plurality of nucleotides. The polymerase may be for sequentially adding nucleotides of the plurality to the first polynucleotide using a sequence of the second polynucleotide.

In some examples, the device may include a first electrode configured to be in contact with the first fluid, a second electrode configured to be in contact with the second fluid, and circuitry in operable communication with the first and second electrodes. The circuitry may be configured to detect changes in an electrical characteristic of the aperture responsive to the polymerase sequentially adding nucleotides of the plurality to the first polynucleotide using a sequence of the second polynucleotide.

Some examples herein provide a sequencing method. The sequencing method may include using the circuitry of such a device to detect changes in the electrical characteristic of the aperture responsive to the polymerase sequentially adding nucleotides of the plurality to the first polynucleotide using a sequence of the second polynucleotide.

In some examples, the electrical characteristic of the aperture may include an electrical conductivity of the aperture.

Some examples herein provide a device. The device may include a fluidic well including a barrier. The barrier may include a first side and a second side. The device may include a first fluidic reservoir having a first amount of a salt therein. The device may include a second fluidic reservoir having a second amount of the salt, and an amount of a compound, therein. The device may include at least one fluidic channel for receiving a first solvent in the first fluidic reservoir such that the first solvent dissolves the first amount of the salt to form a first composition. The at least one fluidic channel further may be for contacting the first side of the barrier with the first composition. The at least one fluidic channel further may be for receiving a second solvent in the second fluidic reservoir such that the second solvent dissolves the second amount of the salt and the amount of the compound to form a second composition. The at least one fluidic channel further may be for contacting the second side of the barrier with the second composition. The second concentration of the salt in the second composition may be different than the first concentration of the salt in the first composition so as to generate a first osmotic pressure across the barrier. The concentration of the compound in the second composition may generate a second osmotic pressure across the barrier that opposes and substantially balances the first osmotic pressure.

In some examples, the fluidic well, the first fluidic reservoir, and the second fluidic reservoir are formed in a common substrate. In some examples, the device further includes the first and second solvents.

In some examples, the device further includes a nanopore disposed within the barrier and providing an aperture fluidically coupling the first side to the second side. In some examples, the device further includes the first and second compositions. In some examples, a portion of the salt in the second composition moves from the second side of the barrier to the first side of the barrier through the aperture. In some examples, the compound in the second composition substantially does not move from the second side of the barrier to the first side of the barrier through the aperture. In some examples, the device further includes a polymerase in the second composition or coupled to the nanopore or the barrier. In some examples, the compound stabilizes the polymerase. In some examples, the compound includes a co-factor of the polymerase. In some examples, the device further includes first and second polynucleotides. In some examples, the second composition further includes a plurality of nucleotides, and wherein the polymerase is for sequentially adding nucleotides of the plurality to the first polynucleotide using a sequence of the second polynucleotide.

In some examples, the device may include a first electrode configured to contact the first composition. The device may include a second electrode configured to contact the second composition. The device may include circuitry in operable communication with the first and second electrodes and configured to detect changes in an electrical characteristic of the aperture that are responsive to the polymerase sequentially adding nucleotides of the plurality to the first polynucleotide using a sequence of the second polynucleotide.

Some examples herein provide a sequencing method. The sequencing method may include using the circuitry of such a device to detect changes in the electrical characteristic of the aperture that are responsive to the polymerase sequentially adding nucleotides of the plurality to the first polynucleotide using a sequence of the second polynucleotide.

Some examples herein provide a method of osmotically balancing a barrier. The method may include contacting a first side of a barrier with first composition including a first concentration of a salt. The method may include contacting a second side of the barrier with a second composition including (i) a second concentration of the salt, and (ii) a concentration of a compound other than the salt. The method may include generating a first osmotic pressure across the barrier using a difference between the first and second concentrations of the salt. The method may include generating a second osmotic pressure across the barrier using the concentration of the compound. The second osmotic pressure may oppose and substantially balance the first osmotic pressure.

In some examples, a nanopore provides an aperture fluidically coupling the first side to the second side.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates a cross-sectional view of an example composition and device including an osmotically balanced barrier.

FIG. 2 schematically illustrates a cross-sectional view of an example use of the composition and device of FIG. 1.

FIG. 3 schematically illustrates a cross-sectional view of another example use of the composition and device of FIG. 1.

FIG. 4 schematically illustrates a cross-sectional view of another example use of the composition and device of FIG. 1.

FIG. 5 schematically illustrates a cross-sectional view of another example use of the composition and device of FIG. 1.

FIG. 6 illustrates a flow of operations in an example method for osmotically balancing a barrier.

FIGS. 7A-7C schematically illustrate example devices for which osmotic and electrical properties were characterized.

FIGS. 8A-8C illustrate plots of the measured normalized capacitance as a function of time for the membranes described with reference to FIG. 7B.

FIGS. 9A-9B illustrate plots of the measured salt concentration and normalized capacitance for the membranes described with reference to FIG. 7B.

FIGS. 10A-10B schematically illustrate changes to the membranes described with reference to FIG. 7B during the measurements described with reference to FIGS. 9A-9B.

FIGS. 11A-11B illustrate plots of the measured normalized capacitance as a function of time for the membranes described with reference to FIGS. 7B and 7C.

FIG. 12 illustrates a plot of the measured normalized number of membranes as a function of time for the membranes described with reference to FIGS. 7B and 7C.

FIGS. 13A-13B illustrate plots of measured current and voltage as a function of time for the devices described with reference to FIGS. 7C and 7B.

FIGS. 14A-14B schematically illustrate plan and cross-sectional views of further details of one nonlimiting example of the nanopore composition and device of FIG. 1.

FIG. 15 schematically illustrates an alternative barrier that may be used in the example described with reference to FIGS. 14A-14B.

FIG. 16 schematically illustrates another alternative barrier that may be used in the example described with reference to FIGS. 14A-14B.

FIG. 17 schematically illustrates a cross-sectional view of another example use of the composition and device of FIG. 1.

### DETAILED DESCRIPTION

Devices including osmotically balanced barriers, and methods of making and using the same, are provided herein.

For example, nanopore sequencing may utilize a nanopore that is inserted into a barrier, and that includes an aperture through which ions and/or other molecules may flow from one side of the barrier to the other. Circuitry may be used to detect a sequence, for example of nucleotides, e.g., during sequencing-by-synthesis (SBS) in which, on a first side of the barrier, a polymerase adds the nucleotides to a growing polynucleotide in an order that is based on the sequence of a template polynucleotide to which the growing polynucleotide is hybridized. The sensitivity of the circuitry may be improved by using a relatively high salt concentration on the second side of the barrier, e.g., so as to enhance electron transport. Such a high salt concentration may reduce or inhibit the activity of the polymerase, so it may be desirable to have a lower salt concentration on the first side of the barrier than on the second side of the barrier. However, the resulting difference in salt concentrations may generate an osmotic pressure that may weaken the barrier, and thus increase the likelihood that the barrier may break or leak during normal use.

As provided herein, a barrier may be stabilized by using two counter-acting osmotic forces that promote both (i) activity of polymerase to incorporate nucleotides into a polynucleotide, and (ii) detection of such nucleotides using circuitry. It will be appreciated, however, that the present barriers are not limited to use with sequencing polynucleotides.

First, some terms used herein will be briefly explained. Then, some example devices including osmotically balanced barriers, and methods of making and using the same, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, device, or system, the term “comprising” means that the compound, composition, device, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to +0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′0 terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly (vinyl chloride), polypropylene, nylons, polyesters, polycarbonates, and poly (methyl methacrylate). Example plastics materials include poly (methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

As used herein, the term “electrode” is intended to mean a solid structure that conducts electricity. Electrodes may include any suitable electrically conductive material, such as gold, palladium, silver, or platinum, or combinations thereof. In some examples, an electrode may be disposed on a substrate. In some examples, an electrode may define a substrate.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules such as amino acids or nucleotides. The nanopore can be disposed within a barrier, or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. nanopores include biological nanopores, solid-state nanopores, or biological and solid-state hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an a-helix bundle nanopore and a B-barrel nanopore as well as all others well known in the art. Example polypeptide nanopores include aerolysin, a-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, CsgG, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105:20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Norcadia farcinica, and lysenin. For further details regarding lysenin, see PCT Publication No. WO 2013/153359, the entire contents of which are incorporated by reference herein.

A “polynucleotide nanopore” is intended to mean a nanopore that is made from one or more nucleic acid polymers. A polynucleotide nanopore can include, for example, a polynucleotide origami.

A “solid-state nanopore” is intended to mean a nanopore that is made from one or more materials that are not of biological origin. A solid-state nanopore can be made of inorganic or organic materials. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state membrane, e.g., a membrane including any such material(s).

A “biological and solid-state hybrid nanopore” is intended to mean a hybrid nanopore that is made from materials of both biological and non-biological origins. Materials of biological origin are defined above and include, for example, polypeptides and polynucleotides. A biological and solid-state hybrid nanopore includes, for example, a polypeptide-solid-state hybrid nanopore and a polynucleotide-solid-state nanopore.

As used herein, a “barrier” is intended to mean a structure that normally inhibits passage of molecules from one side of the barrier to the other side of the barrier. The molecules for which passage is inhibited can include, for example, ions or water soluble molecules such as nucleotides and amino acids. However, if a nanopore is disposed within a barrier, then the aperture of the nanopore may permit passage of molecules from one side of the barrier to the other side of the barrier. As one specific example, if a nanopore is disposed within a barrier, the aperture of the nanopore may permit passage of molecules from one side of the barrier to the other side of the barrier. Barriers include membranes of biological origin, such as lipid bilayers, and non-biological barriers such as solid-state membranes or substrates.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, “synthetic” refers to a membrane material that is not of biological origin (e.g., polymeric materials, synthetic phospholipids, solid-state membranes, or combinations thereof).

As used herein, a “solution” is intended to refer to a homogeneous mixture including two or more substances. In such a mixture, a solute is a substance which is dissolved in another substance referred to as a solvent. A solution may include a single solute, or may include a plurality of solutes. An “aqueous solution” refers to a solution in which the solvent is, or includes, water.

As used herein, the term “osmotic pressure” is intended to refer to the minimum pressure which needs to be applied to a solution to prevent the inward flow of its pure solvent across a semipermeable membrane. “Osmotic pressure” also refers to the measure of the tendency of a solution to take in a pure solvent by osmosis. Potential osmotic pressure is the maximum osmotic pressure that could develop in a solution if it were separated from its pure solvent by a semipermeable membrane. The osmotic pressure of a solution is based, at least in part, on the respective concentration(s) of solute(s) within that solution.

As used herein, a “polymeric membrane” refers to a synthetic barrier that primarily is composed of a polymer that is not of biological origin. In some examples, a polymeric membrane consists essentially of a polymer that is not of biological origin. A block copolymer is an example of a polymer that is not of biological origin and that may be included in the present barriers. When the present barriers relate to polymers that are not of biological origin, the terms “polymeric membrane,” “membrane,” and “barrier” may be used interchangeably herein when referring to the present barriers, even though the terms “barrier” and “membrane” generally may encompass other types of materials as well.

As used herein, terms such as “opposing” are intended to mean in the opposite direction. For example, a first osmotic pressure that is in the opposite direction across a barrier as a second osmotic pressure across that barrier may be said to “oppose” the second osmotic pressure.

As used herein, terms such as “substantially balancing” are intended to mean approximately equal to. For example, a first osmotic pressure that “substantially balances” a second osmotic pressure may be approximately equal to the second osmotic pressure. In examples in which a first osmotic pressure across a barrier opposes, and substantially balances, a second osmotic pressure across that barrier, the first and second osmotic pressures together may exert substantially no net force that would cause the barrier to deform. Such a barrier may be said to be “osmotically balanced.”

As used herein, the term “barrier support” is intended to refer to a structure that can suspend a barrier. A barrier support may define an aperture, such that a first portion of the barrier is suspended across the aperture, and a second portion of the barrier is disposed on, and supported by, the barrier. The barrier support may include any suitable arrangement of elements to define an aperture and suspend the barrier across the aperture. In some examples, a barrier support may include a substrate having an aperture defined therethrough, across which aperture the barrier may be suspended. Additionally, or alternatively, the barrier support may include one or more first features (such as one or more lips or ledges of a well within a substrate) that are raised relative to one or more second features (such as a bottom surface of the well), wherein a height difference between (a) the one or more first features and (b) the one or more second features defines an aperture across which a barrier may be suspended. The aperture may have any suitable shape, such as a circle, an oval, a polygon, or an irregular shape. The barrier support may include any suitable material or combination of materials. For example, the barrier support may be of biological origin, or may be solid state. Some examples, the barrier support may include, or may consist essentially of, an organic material, e.g., a curable resin such as SU-8; polytetrafluoroethylene (PTFE), poly (methyl methacrylate) (PMMA), parylene, or the like. Additionally, or alternatively, various examples, the barrier support may include, or may consist essentially of, an inorganic material, e.g., silicon nitride, silicon oxide, or molybdenum disulfide.

As used herein, the term “annulus” is intended to refer to a liquid that is adhered to a barrier support, located within a barrier, and extends partially into an aperture defined by the barrier support. As such, it will be understood that the annulus may follow the shape of the aperture of the barrier, e.g., may have the shape of a circle, an oval, a polygon, or an irregular shape.

### Devices Including Osmotically Balanced Barriers, and Methods of Making and Using the Same

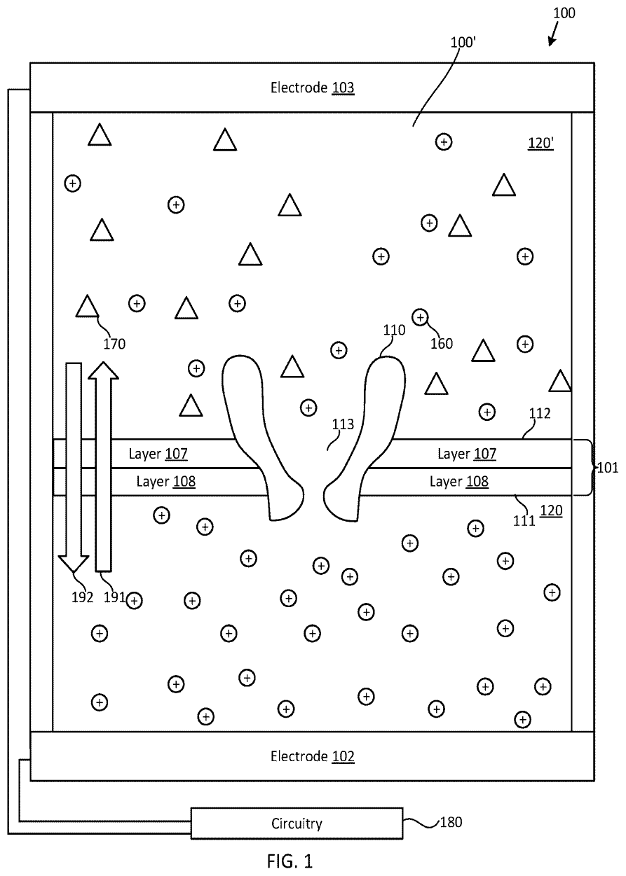

Some example devices including osmotically balanced barriers, and methods of making and using the same, will be described with FIGS. 1, 2, 3, 4, 5, 6, 14A-14B, 15, 16, and 17.

FIG. 1 schematically illustrates a cross-sectional view of an example device 100 including an osmotically balanced barrier. Device 100 includes fluidic well 100′ including barrier 101 having first (trans) side 111 and second (cis) side 112, first fluid 120 within fluidic well 100′ and in contact with first side 111 of the barrier, and second fluid 120′ within the fluidic well and in contact with the second side 112 of the barrier. Barrier 101 may have any suitable structure that normally inhibits passage of molecules from one side of the barrier to the other side of the barrier, e.g., that normally inhibits contact between fluid 120 and fluid 120′. For example, as illustrated in FIG. 1, barrier 101 optionally may include first layer 107 and second layer 108, one or both of which inhibit the flow of molecules across that layer. Illustratively, barrier 101 may include a bilayer including layers 107 and 108, such as lipid layers or polymeric layers. However, it will be appreciated that barrier 101 may include any suitable structure and any suitable number of layers. For example, barrier 101 may include a synthetic barrier, e.g., may include a polymeric membrane.

First fluid 120 may have a first composition including a first concentration of a salt 160, which salt may be represented for simplicity as positive ions although it will be appreciated that counterions also may be present. Second fluid 120′ may have a second composition including a second concentration of the salt 160 that is different than the first concentration. The difference between the first and second concentrations of salt 160 may generate first osmotic pressure 191 across barrier 101. As provided herein, the second composition of second fluid 120′ further may include a concentration of a compound 170 other than the salt 160. The concentration of compound 170 may generate a second osmotic pressure 192 across barrier 101 that opposes and substantially balances the first osmotic pressure 191. For example, the second osmotic pressure 192 across barrier 101, caused by compound 170, may counteract and substantially negate the effect on barrier 101 of first osmotic pressure 191, caused by the difference in concentrations of salt 160 in the first and second compositions. Accordingly, second osmotic pressure 192 may stabilize barrier 101 in a metastable state, e.g., in which the barrier is stabilized by two counter-acting forces. As such, device 100 suitably may be used in repeated cycles of sequencing-by-synthesis (SBS), e.g., in a manner such as described in greater detail below or otherwise known in the art.

Compound 170 may be dissolved in a higher concentration within the second composition of second fluid 120′ than in the first composition of first fluid 120. Indeed, in some examples, the first composition substantially does not include the compound 170. It will be appreciated that the particular magnitudes of the first and second osmotic pressures 191, 192 may be based, at least in part, on the relative concentrations of salt 160 and compound 170 within the first and second compositions. Illustratively, the first concentration of salt 160 (within the first composition of first fluid 120) may be between about 1.1 and about 50 times, or between about 1.5 and about 20 times, or between about 5 and about 20times, or between about 10 and about 20 times, or between about 2 and about 10 times, the second concentration of the salt (within the second composition of second fluid 120′). The concentration of compound 170 (within the second composition of second fluid 120′) may be between about 1.1 and about 50 times the first concentration of the salt 160 (within the first composition of first fluid 120), or between about 0.1 and about 10 times the first concentration of the salt, or between about 0.5 and about 5 times the first concentration of the salt, or between about 1.5 and about 20 times the first concentration of the salt, or between about 2 and about 10 times the first concentration of the salt, or between about 1 and about 5times the concentration of the salt, illustratively about the same concentration as the salt. Additionally, or alternatively, the particular magnitudes of the first and second osmotic pressures 191, 192 may be based, at least in part, on the absolute concentrations of salt 160 and compound 170 within the first and second compositions. Illustratively, the first concentration of the salt 160 (within the first composition of first fluid 120) may be above about 150 mM, or above about 250 mM, and the second concentration of the salt 160 (within the second composition of second fluid 120′) may be below about 100 mM, or below about 50 mM. The concentration of the compound 170 (within the second composition of second fluid 120′) may, for example, be above about 100 mM.

Any suitable salt or salts 160 may be used to generate first osmotic pressure 191, for example any suitable combination of ions in solution, e.g., ranging from common salts to ionic crystals, metal complexes, ionic liquids, or even water soluble organic ions. For example, the salt may include any suitable combination of cations (such as, but not limited to, H, Li, Na, K, NH₄, Ag, Ca, Ba, and/or Mg) with any suitable combination of anions (such as, but not limited to, OH, Cl, Br, I, NO₃, ClO₄, F, SO₄, and/or CO₃²⁻ . . . ). In one nonlimiting example, the salt includes potassium chloride (KCl). Any suitable compound 170 may be used to generate second osmotic pressure 192 that opposes and substantially balances first osmotic pressure 191 and is sufficiently bulky as substantially not to pass through aperture 113 of nanopore. In some examples, compound 170 is charge neutral and does not bear formal charges, e.g., such that compound 170 may be inert to electrical detection during SBS such as disclosed elsewhere herein or otherwise known in the art. Additionally, or alternatively, compound 170 may increase viscosity of the second fluid 120′, which may be useful during SBS. Illustratively, compound 170 may include an alcohol, such as polyethylene glycol (PEG); a protein, such as a recombinase (e.g., UvsY), bovine serum albumin, or polymerase cofactor VP35; or a polysaccharide, such as trehalose or a cyclodextrin. It will be appreciated that the second composition of second fluid 120′ may include more than one such compound 170, e.g., may include two or more of an alcohol, a protein, or a polysaccharide, or any other suitable combination of compounds that generate second osmotic pressure 192 that opposes and substantially balances first osmotic pressure 191.

It will also be appreciated that the first and second compositions optionally may include any suitable combination of other solutes. Illustratively, the first composition of first fluid 120 may include a first concentration of an aqueous buffer (such as N-(2-hydroxyethyl) piperazine-N′-2-ethanesulfonic acid (HEPES), commercially available from Fisher BioReagents). Additionally, or alternatively, the second composition of second fluid 120′ may include a second concentration of the aqueous buffer. The first concentration of the aqueous buffer may be approximately equal to the second concentration of the aqueous buffer.

Still referring to FIG. 1, in some examples provided herein, device 100 optionally further may include nanopore 110 disposed within barrier 101 and providing aperture 113 fluidically coupling first side 111 to second side 112. As such, aperture 113 of nanopore 110 may provide a pathway for fluid 120 and/or fluid 120′ to flow through barrier 101. For example, a portion of salt 160 may move from second side 112 of barrier 101 to first side 111 of the barrier through aperture 113. Additionally, or alternatively, compound 170 substantially may not move from second side 112 of barrier 101 to first side 111 of the barrier through aperture 113. For example, compound 170 may be larger in at least one dimension than aperture 113, or otherwise may be sterically hindered from passing through aperture 113. Nanopore 110 may include a solid-state nanopore, a biological nanopore (e.g., MspA such as illustrated in FIG. 1), or a biological and solid state hybrid nanopore. Nonlimiting examples and properties of barriers and nanopores are described elsewhere herein, as well as in U.S. Pat. No. 9,708,655, the entire contents of which are incorporated by reference herein.

In a manner such as illustrated in FIG. 1, device 100 optionally may include first electrode 102 in contact with first fluid 120, second electrode 103 in contact with second fluid 120′, and circuitry 180 in operable communication with the first and second electrodes and configured to detect changes in an electrical characteristic of the aperture. Such changes may, for example, be responsive to any suitable stimulus. Indeed, it will be appreciated that the present methods, compositions, and devices may be used to osmotically balance barriers for use in any suitable application or context, including any suitable method or device for sequencing, e.g., polynucleotide sequencing.

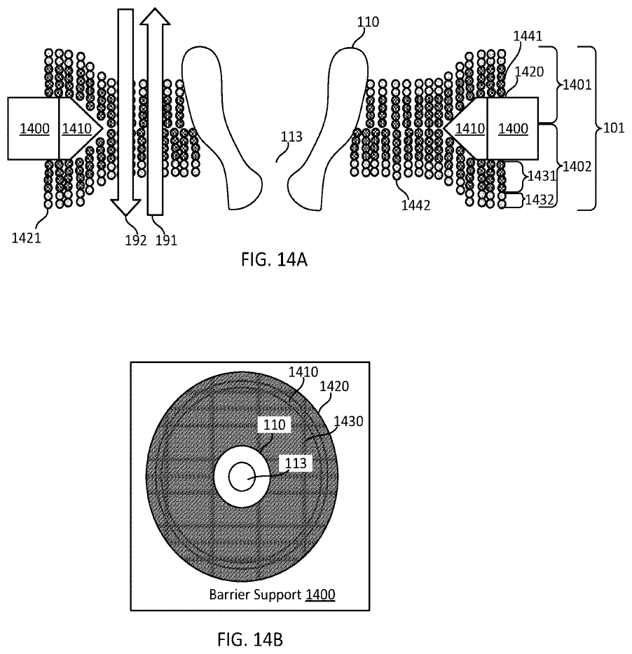

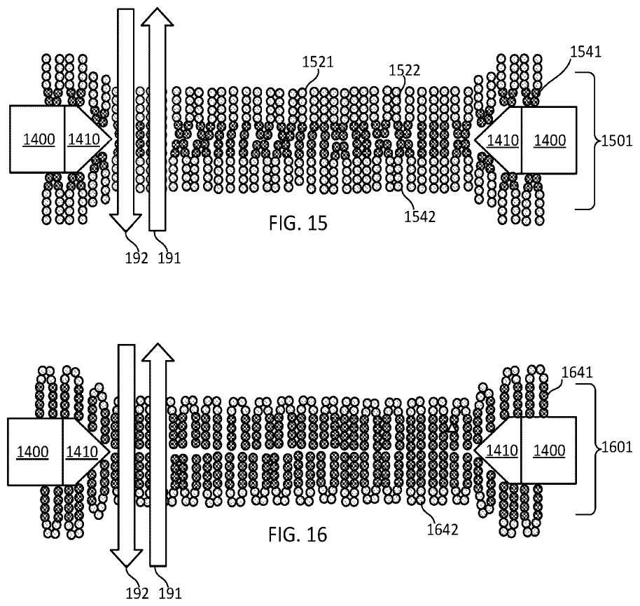

Illustratively, barrier 101 may include a bilayer including layers 107 and 108 which respectively may be formed using an AB diblock copolymer, or an BAB triblock copolymer, or certain ABA triblock copolymer, and may have a structure such as described in greater detail below with reference to FIGS. 14A-14B, 15, or 16. Alternatively, barrier 101 may include only a single layer, which inhibits the flow of molecules across that layer. Illustratively, barrier 101 may include a single layer which may be formed using certain ABA triblock copolymer, and may have a structure such as described in greater detail below with reference to FIG. 15. In other examples, barrier 101 may be partially a single layer, and partially a bilayer, formed using certain ABA triblock copolymer, and may have a structure such as described in greater detail below with reference to FIG. 15.

More specifically, in some examples, first layer 107 of barrier 101 between first and second fluids 120, 120′ includes a first plurality of molecules of a diblock or triblock copolymer, and second layer 108 of barrier 101 includes a second plurality of molecules of the copolymer. In examples in which the copolymer is a diblock copolymer (which may be referred to as AB), each molecule may include a hydrophobic block coupled to a hydrophilic block. In some examples in which the copolymer is a triblock copolymer, each molecule may include first and second hydrophobic blocks and a hydrophilic block disposed therebetween (which polymer may be referred to as BAB). Regardless of whether the copolymer is diblock (AB) or triblock (BAB), the hydrophilic blocks of the first plurality of molecules may form a first outer surface of barrier 101, e.g., the surface of layer 107 contacting fluid 120 on first side 111. The hydrophilic blocks of the second plurality of molecules may form a second outer surface of barrier 101, e.g., the surface of layer 108 contacting fluid 120′ on second side 112. The hydrophobic blocks of the first and second pluralities of molecules may contact one another within the barrier.

In other examples, barrier 101 between first and second fluids 120, 120′ includes a plurality of molecules of a triblock copolymer that includes first and second hydrophilic blocks and a hydrophobic block disposed therebetween (which polymer may be referred to as ABA). The first hydrophilic blocks of the plurality of molecules may form a first outer surface of barrier 101, e.g., the surface of the barrier 101 contacting fluid 120 on first side 111. The second hydrophilic blocks of the plurality of molecules may form a second outer surface of barrier 101, e.g., contacting fluid 120′ on second side 112. The hydrophobic blocks of the plurality of molecules may contact one another within the barrier. In some examples, the ABA molecules that form barrier 101 may be present in a single layer rather than the two layers illustrated in FIG. 1. In other examples, the ABA molecules that form barrier 101 may be present in a bilayer similar to that illustrated in FIG. 1, because the molecules of the B blocks of the molecules are folded such that both A blocks of a given molecule contact the same fluid as one another. In still other examples, a portion of barrier 101 may be a single layer (because the ABA molecules of that portion extend from one fluid to the other fluid) while a different portion of barrier 101 may be a bilayer (because the ABA molecules of that portion are folded so the A blocks of that portion contact the same fluid as one another); FIG. 15, described below, illustrates an example of this.

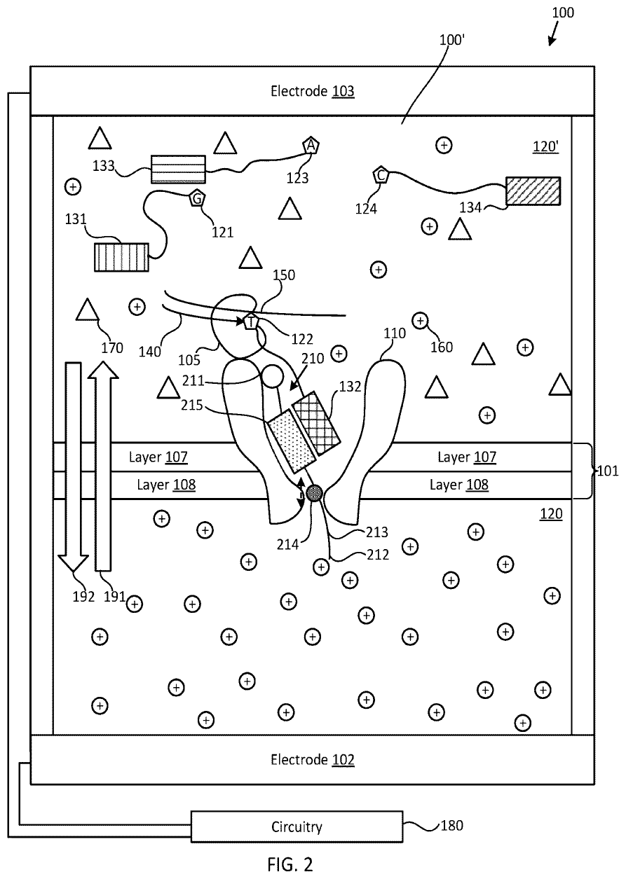

FIGS. 14A-14B schematically illustrate plan and cross-sectional views of further details of one nonlimiting example of the nanopore composition and device of FIG. 1. More specifically, in the example illustrated in FIGS. 14A-14B, barrier 101 may be suspended using barrier support 1400 defining aperture 1430. For example, barrier support 1400 may include a substrate having an aperture 1430 defined therethrough, e.g., a substantially circular aperture. Additionally, or alternatively, the barrier support may include one or more features of a well in which the nanopore device is formed, such as a lip or ledge on either side of the well. Nonlimiting examples of materials which may be included in a barrier support are provided further above. An annulus 1410 including hydrophobic (non-polar) solvent, and which also may include other compound(s), may adhere to barrier support 1400 and may support a portion of barrier 101, e.g., may be located within barrier 101 (here, between layer 1401 and layer 1402). Additionally, annulus 1410 may taper inwards in a manner such as illustrated in FIG. 14A. An outer portion of the molecules 1421 of barrier 101 may be disposed on support 1400 (e.g., the portion extending between aperture 1430 and barrier periphery 1420), while an inner portion of the molecules may form a freestanding portion of barrier 101 (e.g., the portion within aperture 1410, a part of which is supported by annulus 1410). Barrier 101 may be prepared, and nanopore 110 may be inserted into the freestanding portion of barrier 101, using operations such as described elsewhere herein. Although FIGS. 14A-14B illustrate nanopore 110 within barrier 101, it should be understood that the nanopore may be omitted, and that barrier 101 used for any suitable purpose. More generally, it should be appreciated that while the barriers described herein are particularly suitable for use with nanopores (e.g., for nanopore sequencing such as described with reference to FIGS. 2-5 and 17), the present barriers need not necessarily have nanopores inserted therein.

In the nonlimiting example illustrated in FIG. 14A, barrier 101 may include first layer 1401 including a first plurality of amphiphilic molecules 1421 and second layer 1402 including a second plurality of the amphiphilic molecules contacting the first plurality of amphiphilic molecules. In the nonlimiting example illustrated in FIG. 14A, the copolymer is a diblock copolymer (AB). Here, each molecule 1421 includes a hydrophobic “B” block 1431 (within which circles 1441 with darker fill represent hydrophobic monomers) and a hydrophilic “A” block 1432 (within which circles 1442 with lighter fill represent hydrophilic monomers) coupled directly or indirectly thereto. In other examples such as will be described with reference to FIGS. 15 and 16, the copolymer instead may include a triblock copolymer (e.g., ABA or BAB, respectively). In the example illustrated in FIG. 14A, the hydrophilic blocks 1432 of the first plurality of molecules 1421 may form a first outer surface of barrier 101, e.g., the surface of barrier 101 contacting fluid 120 on first side 111. The hydrophilic blocks 1432 of the second plurality of molecules 1421 may form a second outer surface of barrier 101, e.g., the surface of barrier 101 contacting fluid 120′ on second side 112. The hydrophobic blocks 1431 of the first and second pluralities of molecules 1421 may contact one another within the barrier.

Although FIGS. 14A-14B illustrate a suspended barrier that includes a diblock copolymer, it will be appreciated that suspended barriers that included other types of polymers provided herein, are similarly contemplated. FIG. 15 schematically illustrates an alternative barrier that may be used in the example described with reference to FIGS. 14A-14B. FIG. 15 illustrates barrier 1501 which is suspended using barrier support 1400 and annulus 1410 in a manner such as described with reference to FIGS. 14A-14B. In this example, barrier 1501 includes molecules of an ABA triblock copolymer such as described with reference to FIG. 2A. Here, the triblock copolymer includes hydrophobic “B” sections 1541 coupled to and between hydrophilic “A” sections 1542. In the example shown in FIG. 15, each individual ABA molecule may be in one of two arrangements. For example, ABA molecules 1521 may extend through the layer in a linear fashion, with an “A” section on each side of the barrier and the “B” section in the middle of the barrier. Or, for example, ABA molecules 1522 may extend to the middle of the barrier and then fold back on themselves, so that both “A” sections are on the same side of the barrier and the “B” section is in the middle of the barrier. Accordingly, in this example, barrier 1501 may be considered to be partially a single layer and partially a bilayer. In other examples (not specifically illustrated) in which barrier 1501 substantially includes molecules 1521 which extend through the barrier in linear fashion, barrier 1501 may substantially be a monolayer. In still other examples (not specifically illustrated) in which barrier 1501 substantially includes molecules 1522 extend to approximately the middle of the barrier and then fold back on themselves, barrier 1501 may substantially be a bilayer. A nanopore, not specifically, shown, optionally may be inserted into any of such options for barrier 1501 in a manner similar to that described elsewhere herein, e.g., as illustrated in FIGS. 14A-14B.

FIG. 16 schematically illustrates an alternative barrier that may be used in the example described with reference to FIGS. 14A-14B. FIG. 16 illustrates barrier 1601 which is suspended using barrier support 1400 and annulus 1410 in a manner such as described with reference to FIGS. 14A-14B. In this example, barrier 1601 includes molecules of a BAB triblock copolymer. Here, the triblock copolymer includes hydrophilic “A” sections 1642 coupled to and between hydrophobic “B” sections 1641. In this example, barrier 1601 may have a bilayer architecture with the “B” sections 1641 oriented towards each other. The hydrophobic ends of the BAB molecules generally may located approximately in the middle of barrier 1601, the molecules then extend towards either outer surface of the barrier, and then fold back on themselves. As such, both “B” sections are located in the middle of the barrier and the “A” section is on one side or the other of the barrier. A nanopore, not specifically, shown, optionally may be inserted into barrier 1601 in a manner similar to that described elsewhere herein, e.g., as illustrated in FIGS. 14A-14B.

It will be appreciated that nanopore devices such as described with reference to FIG. 1 may be made using any suitable barriers such as, but not limited to, those described with reference to FIGS. 14A-14B, 15, and 16. Additionally, the barriers may be made using any suitable copolymers. For example, device 100 described with reference to FIG. 1 may be made using operations that include forming the barrier in the fluidic well; and inserting the nanopore within the barrier. The barrier may be suspended using a barrier support 1400 in a manner such as described with reference to FIGS. 14A-14B, 15, and 16. Forming the barrier may include “painting” as known in the art. Known techniques for painting barriers that are suspended by barrier supports include brush painting (manual), mechanical painting (e.g., using stirring bar), and bubble painting (e.g., using flow through the device). Known techniques for inserting a nanopore into a suspended barrier include electroporation, pipette pump cycle, and detergent assisted pore insertion. Tools for forming suspended barriers using synthetic polymers and inserting nanopores in the suspended barriers are commercially available, such as the Orbit 16 TC platform available from Nanion Technologies Inc. (California, USA).

It will further be appreciated that the present barriers may be used in any suitable device or application. For example, FIG. 2 schematically illustrates a cross-sectional view of an example use of the composition and device of FIG. 1. Device 100 illustrated in FIG. 2 may be configured may include fluidic well 100′, barrier 101, first and second fluids 120, 120′, and nanopore 110 in a manner such as described with reference to FIG. 1, 14A-14B, 15, and/or 16 (that is, barrier 101 optionally may be suspended using a barrier support, and may include any suitable AB, ABA, or BAB copolymer. In the nonlimiting example illustrated in FIG. 2, the second composition of second fluid 120′ optionally may include a plurality of each of nucleotides 121, 122, 123, 124, e.g., G, T, A, and C, respectively. Each of the nucleotides 121, 122, 123, 124 in the second composition optionally may be coupled to a respective label 131, 132, 133, 134 coupled to the nucleotide via an elongated body (elongated body not specifically labeled). Optionally, device 100 further may include polymerase 105. As illustrated in FIG. 2, polymerase 105 may be within the second composition of second fluid 120′. Alternatively, polymerase 105 may be coupled to 110 nanopore or to barrier 101, e.g., via a suitable elongated body (not specifically illustrated). In examples in which polymerase 105 is included, compound 170 may stabilize the polymerase and/or may include a co-factor of the polymerase. For example, polymerases' performances are known to vary with the composition of the solution they are in. Illustratively, the presence of PEG in solution may improve polymerase activity. Compound 170 may be selected not only to counter-act the effect of osmotic pressure and thus stabilize barrier 101, but also may be beneficial (like a cofactor) to the activity of the enzyme itself, for example by preserving the polymerase and making it more robust and extending the shelf life, or by improving the base incorporation.

Device 100 optionally further may include first and second polynucleotides 140, 150 in a manner such as illustrated in FIG. 2. Polymerase 105 may be for sequentially adding nucleotides of the plurality to the first polynucleotide 140 using a sequence of the second polynucleotide 150. For example, at the particular time illustrated in FIG. 2, polymerase 105 incorporates nucleotide 122 (T) into first polynucleotide 140, which is hybridized to second polynucleotide 150 to form a duplex. At other times (not specifically illustrated), polymerase 105 sequentially may incorporate other of nucleotides 121, 122, 123, 124 into first polynucleotide 140 using the sequence of second polynucleotide 150.

Circuitry 180 illustrated in FIG. 2 may be configured to detect changes in an electrical characteristic of the aperture responsive to the polymerase sequentially adding nucleotides of the plurality to the first polynucleotide 140 using a sequence of the second polynucleotide 150. In the nonlimiting example illustrated in FIG. 2, nanopore 110 may be coupled to permanent tether 210 which may include head region 211, tail region 212, elongated body 213, reporter region 214 (e.g., an abasic nucleotide), and moiety 215. Head region 211 of tether 210 is coupled to nanopore 110 via any suitable chemical bond, protein-protein interaction, or any other suitable attachment that is normally irreversible. Head region 211 can be attached to any suitable portion of nanopore 110 that places reporter region 214 within aperture 213 and places moiety 215 sufficiently close to polymerase 105 so as to interact with respective labels 131, 132, 133, 134 of nucleotides 121, 122, 123, 124 that are acted upon by polymerase 105. Moiety 215 respectively may interact with labels 131, 132, 133, 134 in such a manner as to move reporter region 214 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to nanopores to sequence polynucleotides, see U.S. Pat. No. 9,708,655, the entire contents of which are incorporated by reference herein.

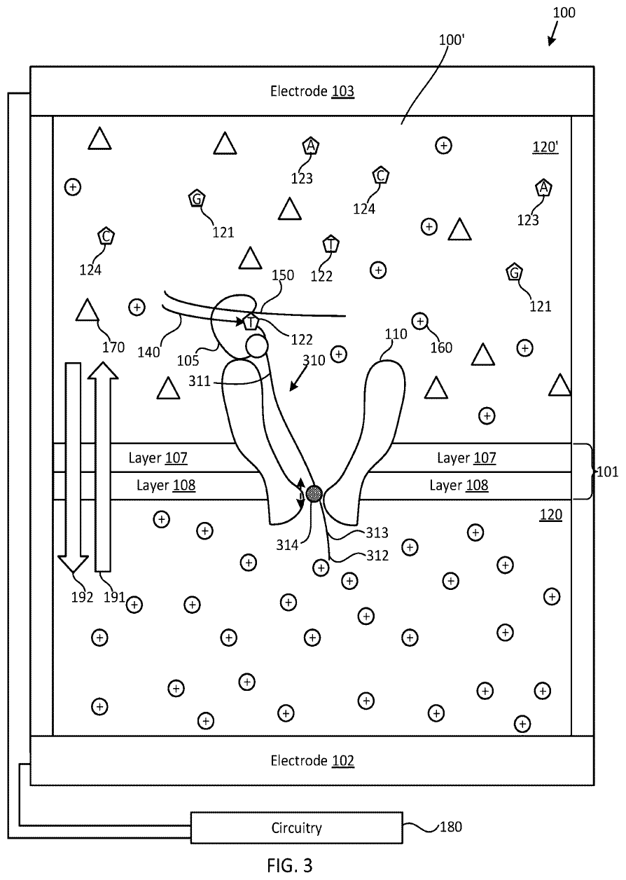

FIG. 3 schematically illustrates a cross-sectional view of another example use of the composition and device of FIG. 1. As illustrated in FIG. 3, device 100 may include fluidic well 100′, barrier 101 which may have a configuration such as described with reference to FIGS. 14A-14B, 15, and/or 16 (that is, barrier 101 optionally may be suspended using a barrier support, and may include any suitable AB, ABA, or BAB copolymer), first and second fluids 120, 120′, nanopore 110, and first and second polynucleotides 140, 150, all of which may be configured similarly as described with reference to FIG. 2. In the nonlimiting example illustrated in FIG. 3, nucleotides 121, 122, 123, 124 need not necessarily be coupled to respective labels. Polymerase 105 may be coupled to nanopore 110 and may be coupled to permanent tether 310 which may include head region 311, tail region 312, elongated body 313, and reporter region 314 (e.g., an abasic nucleotide. Head region 311 of tether 310 is coupled to polymerase 105 via any suitable chemical bond, protein-protein interaction, or any other suitable attachment that is normally irreversible. Head region 311 can be attached to any suitable portion of polymerase 105 that places reporter region 314 within aperture 113. As polymerase 105 interacts with nucleotides 121, 122, 123, 124, such interactions may cause polymerase 105 to undergo conformational changes. Such conformational changes may move reporter region 314 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to polymerases to sequence polynucleotides, see U.S. Pat. No. 9,708,655, the entire contents of which are incorporated by reference herein.

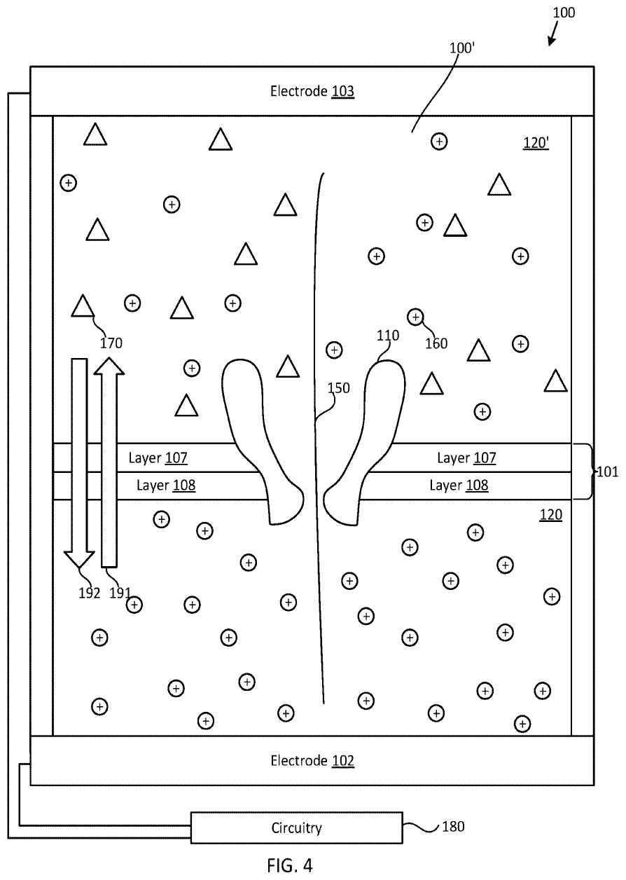

FIG. 4 schematically illustrates a cross-sectional view of another example use of the composition and device of FIG. 1. As illustrated in FIG. 4, device 100 may include fluidic well 100′, barrier 101 which may have a configuration such as described with reference to FIGS. 14A-14B, 15, and/or 16 (that is, barrier 101 optionally may be suspended using a barrier support, and may include any suitable AB, ABA, or BAB copolymer), first and second fluids 120, 120′, and nanopore 110 all of which may be configured similarly as described with reference to FIG. 2. In the nonlimiting example illustrated in FIG. 4, polynucleotide 150 is translocated through nanopore 110 under an applied force, e.g., a bias voltage that circuitry applies between electrode 102 and electrode 103. As bases in polynucleotide 150 pass through nanopore 110, such bases may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see U.S. Pat. No. 5,795,782, the entire contents of which are incorporated by reference herein.

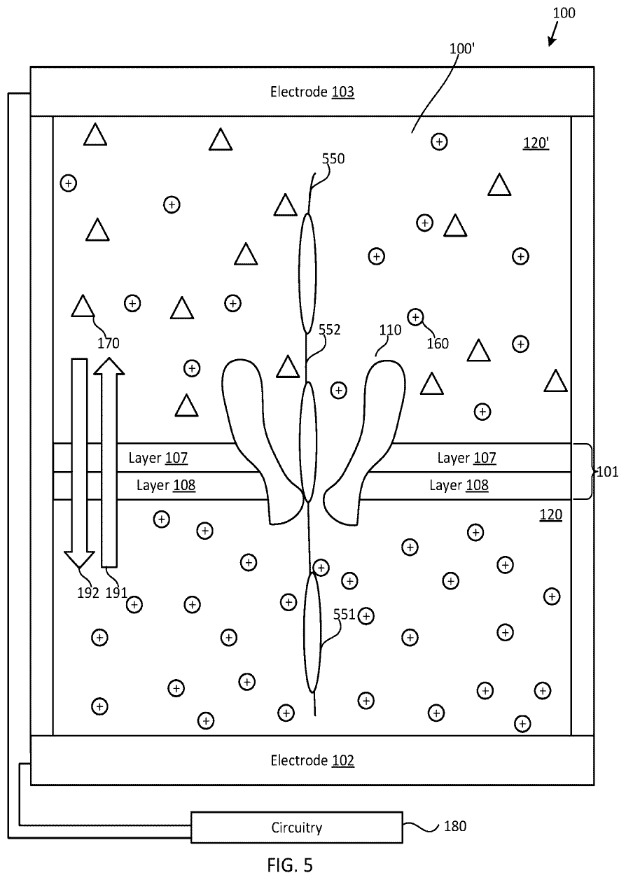

FIG. 5 schematically illustrates a cross-sectional view of another example use of the composition and device of FIG. 1. As illustrated in FIG. 5, device 100 may include fluidic well 100′, barrier 101 which may have a configuration such as described with reference to FIGS. 14A-14B, 15, and/or 16 (that is, barrier 101 optionally may be suspended using a barrier support, and may include any suitable AB, ABA, or BAB copolymer), first and second fluids 120, 120′, and nanopore 110 all of which may be configured similarly as described with reference to FIG. 2. In the nonlimiting example illustrated in FIG. 5, surrogate polymer 550 is translocated through nanopore 110 under an applied force, e.g., a bias voltage that circuitry 180 applies between electrode 102 and electrode 103. As used herein, a “surrogate polymer” is intended to mean an elongated chain of labels having a sequence corresponding to a sequence of nucleotides in a polynucleotide. In the example illustrated in FIG. 5, surrogate polymer 550 includes labels 551 coupled to one another via linkers 552. An XPANDOMER™ is a particular type of surrogate polymer developed by Roche Sequencing, Inc. (Pleasanton, CA). XPANDOMERS™ may be prepared using Sequencing By expansion™ (SBX™, Roche Sequencing, Pleasanton CA). In Sequencing by expansion™, an engineered polymerase polymerizes xNTPs which include nucleobases coupled to labels via linkers, using the sequence of a target polynucleotide. The polymerized nucleotides are then processed to generate an elongated chain of the labels, separated from one another by linkers which are coupled between the nucleotides, and having a sequence that is complementary to that of the target polynucleotide. For example descriptions of XPANDOMERS™, linkers (tethers), labels, engineered polymerases, and methods for SBX™, see the following patents, the entire contents of each of which are incorporated by reference herein: U.S. Pat. Nos. 7,939,249, 8,324,360, 8,349,565, 8,586,301, 8,592,182, 9,670,526, 9,771,614, 9,920,386, 10,301,345, 10,457,979, 10,676,782, 10,745,685, 10,774,105, and 10,851,405.

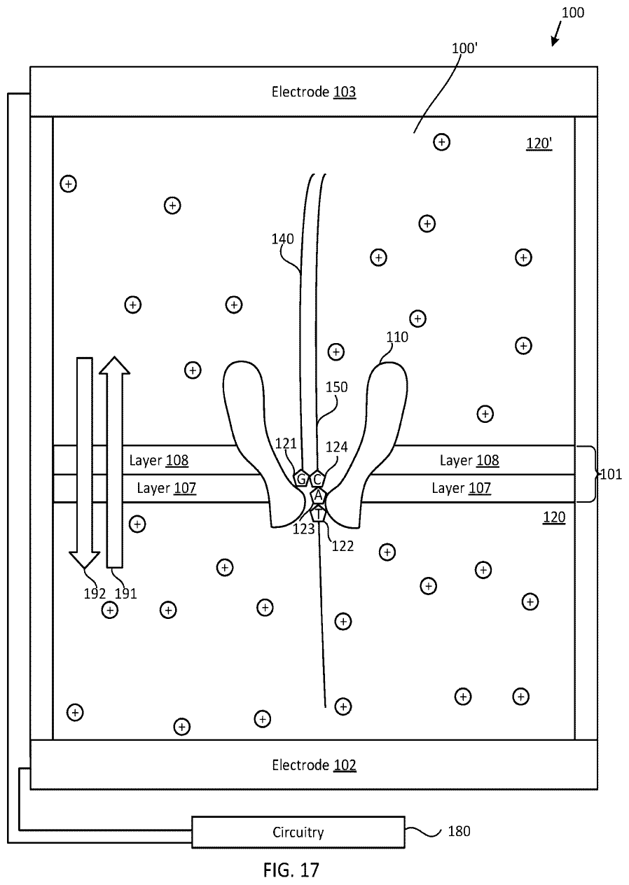

FIG. 17 schematically illustrates a cross-sectional view of another example use of the composition and device of FIG. 1. As illustrated in FIG. 17, device 100 may include fluidic well 100′, barrier 101 which may have a configuration such as described with reference to FIGS. 2A-2C, 14A-14B, 15, and/or 16 (that is, barrier 101 optionally may be suspended using a barrier support, and may include any suitable AB, ABA, or BAB copolymer), first and second fluids 120, 120′, and nanopore 110 all of which may be configured similarly as described with reference to FIG. 4. In the nonlimiting example illustrated in FIG. 17, a duplex between polynucleotide 140 and polynucleotide 150 is located within nanopore 110 under an applied force, e.g., a bias voltage that circuitry 180 applies between electrode 102 and electrode 103. A combination of bases in the double-stranded portion (here, the base pair GC 121, 124 at the terminal end of the duplex) and bases in the single-stranded portion of polynucleotide 150 (here, bases A and T 123, 122) may alter the rate at which salt 160 moves through aperture 113, and thus may detectably alter the electrical conductivity of aperture 113 in such a manner as to be detected by circuitry 180. For further details regarding use of nanopores for sequencing, see US Patent Publication No. 2023/0090867 to Mandell et al., the entire contents of which are incorporated by reference herein.

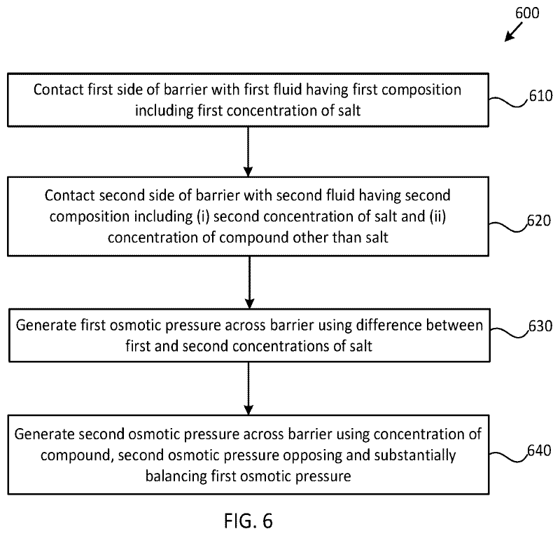

Barriers such as described with reference to FIGS. 1-5, 14A-14B, 15, 16, and 17 may be osmotically balanced using any suitable combination of operations provided herein. FIG. 6 illustrates a flow of operations in an example method 600 for osmotically balancing a barrier. As illustrated in FIG. 6, method 600 may include contacting a first side of a barrier with a first fluid having a first composition including a first concentration of a salt (operation 610). For example, first side 111 of barrier 101 may be contacted with fluid 120 having a first composition including a first concentration of salt 160 in a manner such as described with reference to FIG. 1. As illustrated in FIG. 6, method 600 may include contacting a second side of the barrier with a second fluid having a second composition including (i) a second concentration of the salt, and (ii) a concentration of a compound other than the salt (operation 620). For example, second side 112 of barrier 101 may be contacted with fluid 120′ in a manner such as described with reference to FIG. 1. As illustrated in FIG. 6, method 600 may include generating a first osmotic pressure across the barrier using a difference between the first and second concentrations of the salt (operation 630). For example, the difference between the first concentration of salt 160 in the first composition of fluid 120 and the second concentration of salt in the second composition of fluid 120′ may generate osmotic pressure 191. As illustrated in FIG. 6, method 600 may include generating a second osmotic pressure across the barrier using the concentration of the compound, the second osmotic pressure opposing and substantially balancing the first osmotic pressure (operation 640). For example, the concentration of compound 170 in the second composition of fluid 120′ may generate osmotic pressure 192. Osmotic pressure 192 may opposed, and substantially balance, osmotic pressure 191. As such, barrier 101 may be relatively stable, e.g., may be sufficiently stable for prolonged use (e.g., for hours or days) of barrier 101 during sequencing operations, e.g., polynucleotide sequencing operations, that optionally may use nanopore 101 including aperture 113 fluidically coupling first side 111 to second side 112. It will be appreciated that operations 610, 620, 630, 640 may be performed in any suitable order, and are not limited to the particular order suggested in FIG. 6.

### WORKING EXAMPLES

The following examples are intended to be purely illustrative, and not limiting of the present invention.

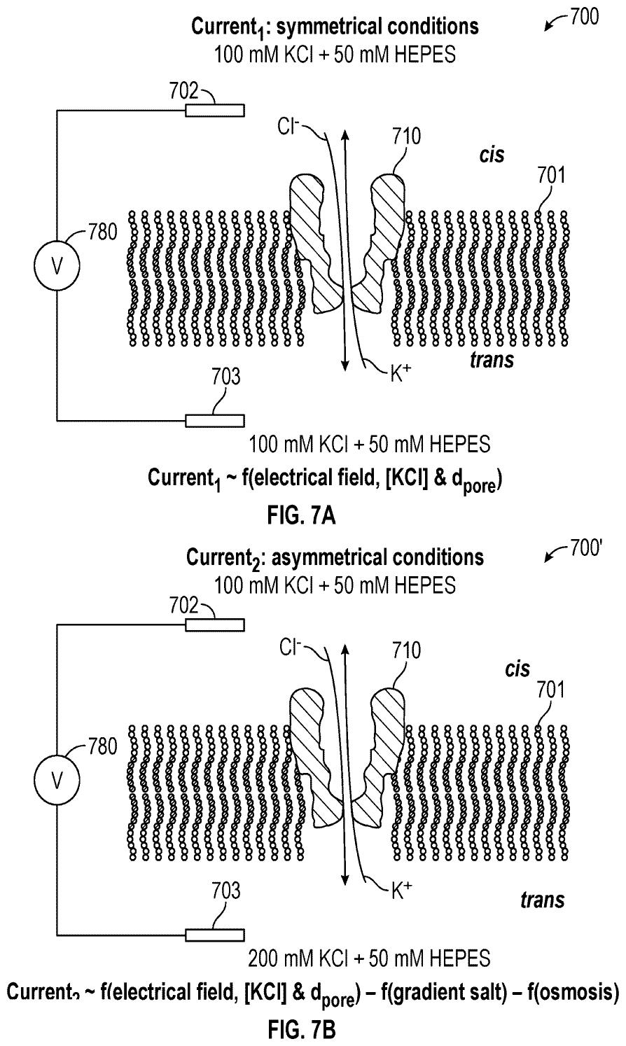

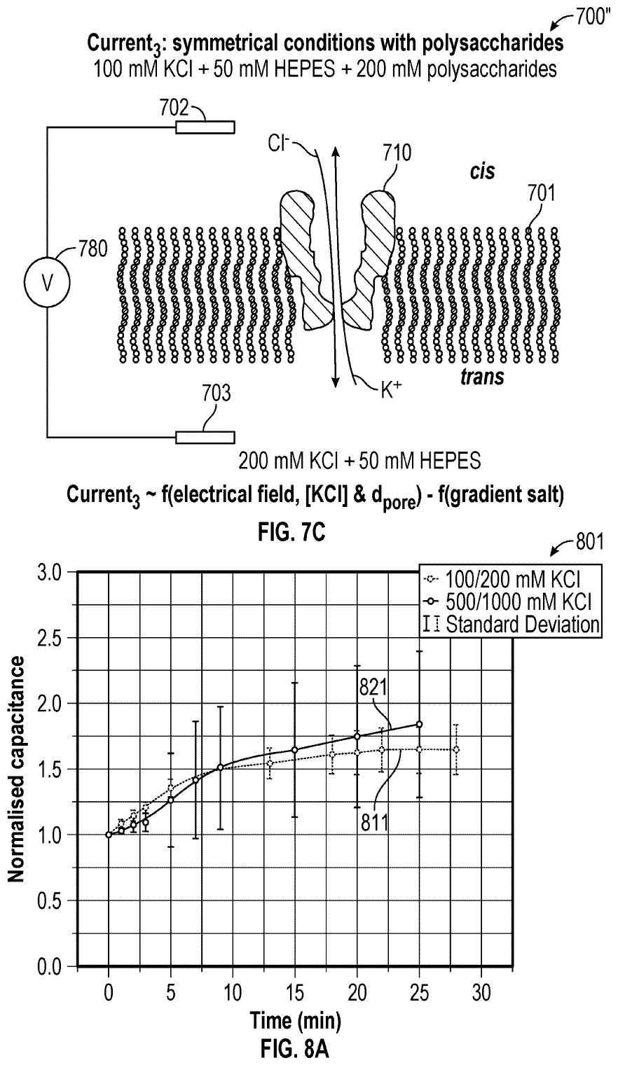

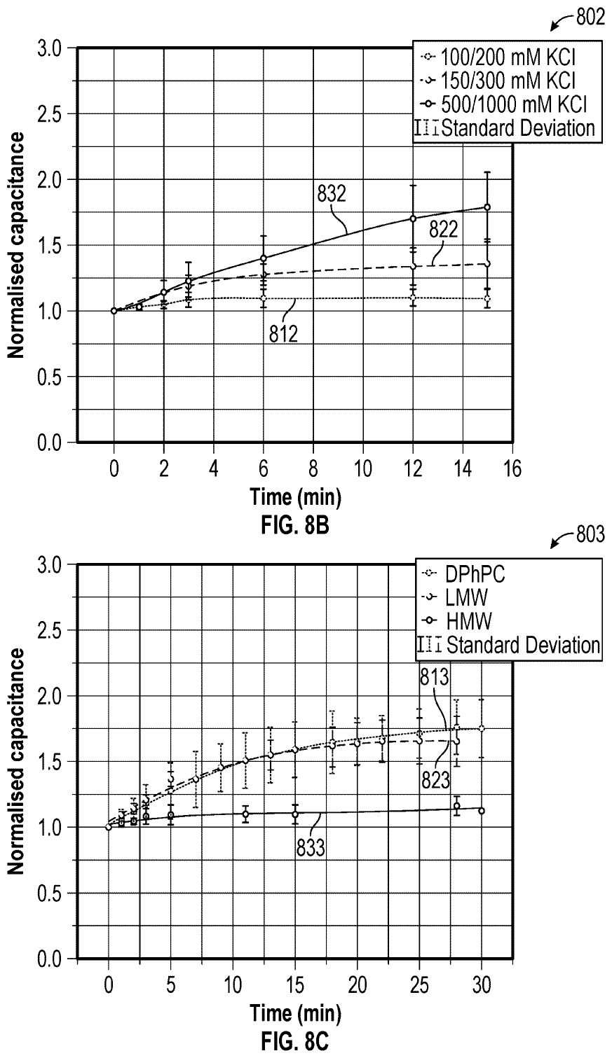

FIGS. 7A-7C schematically illustrate example devices for which osmotic and electrical properties were characterized in a manner such as described with reference to FIGS. 8A-8C, 9A-9B, 10A-10B, 11A-11B, 12, and 13A-13B. Devices 700, 700′, 700″ respectively illustrated in FIGS. 7A, 7B, and 7C each optionally include MspA nanopore 710 within semipermeable membrane 701. In the present examples, semipermeable membrane 701 included a bilayer of either the phospholipid dipalmitoylphosphatidylcholine (DPhPC), a low molecular weight diblock amphiphilic copolymer (referred to as LMW), or a high molecular weight diblock copolymer (referred to as HMW). In “symmetrical” device 700 illustrated in FIG. 7A, the liquid on the first (cis) side of membrane 701 has approximately the same composition as the liquid on the second (trans) side of the membrane, illustratively 100 mM KCl+50 mM HEPES in water on both the cis and trans sides. In “asymmetrical” device 700′ illustrated in FIG. 7B, the liquid on the first side of membrane 101 has a different composition than the liquid on the second side of the membrane, without the use of a compound to balance the osmotic pressure that the asymmetry applies to the membrane, illustratively 100 mM KCl+50 mM HEPES in water on the cis side and 200 mM KCl+50mM HEPES in water on the trans side. In “asymmetrical” device 700″ illustrated in FIG. 7C, the liquid on the first side of membrane 701 has a different composition than the liquid on the second side of the membrane, and further includes a compound to balance the osmotic pressure that the asymmetry applies to the membrane, illustratively 100 mM KCl+50 mM HEPES+200 mM polysaccharides in water on the cis side and 200 mM KCl+50 mM HEPES in water on the trans side.

Devices 700, 700′, 700″ may exhibit different currents in response to a voltage that circuitry 780 applies across electrode 702 (in contact with the liquid on the cis side) and electrode 703 (in contact with the liquid on the trans side). For example, Current₂ exhibited by device 700 may be expected to be a function of the electrical field, concentration of KCl on the trans side, and inner diameter of the nanopore. Current₂ exhibited by device 700′ may be expected to be a positive function of the electrical field, concentration of KCl on the trans side, and inner diameter of the nanopore, as well as a negative function of the salt gradient and a negative function of the membrane's osmosis (e.g., caused by deformation resulting from the salt gradient). Current₃ exhibited by device 700″ may be expected to be a positive function of the electrical field, concentration of KCl on the trans side, and inner diameter of the nanopore, as well as a negative function of the salt gradient. Because the cis side liquid of device 700″ includes polysaccharides or other suitable compound to substantially balance the osmotic pressure across membrane 701, any function in Current₃ relating to the membrane's osmosis (e.g., caused by deformation resulting from the salt gradient) is expected to be approximately zero, and thus is ignored. Current₁ is expected to be significantly larger than Current₂ because the negative functions of the salt gradient and of the membrane's osmosis are expected to at least partially counterbalance the positive function of the electrical field, concentration of KCl, and inner diameter of the nanopore. Current₃ is expected to be larger than Current₂ because it lacks the negative function of the membrane's osmosis (e.g., caused by deformation resulting from the salt gradient). In another examples, Current₁ (control condition) corresponds to an under 100 mM symmetrical condition (low salt concentration to maintain polymerase activity, but low resulting current), Current₂ corresponds to an 100 mM Cis/500 mM or 1000 mM Trans asymmetrical condition (low salt in cis to maintain polymerase activity, high salt in trans to boost current, osmosis limiting current boost), and Current₃ corresponds to 100 mM+polysaccharide cis/500 mM or 1000 mM trans asymmetrical condition (low salt in cis to maintain polymerase activity+polysaccharide to prevent osmosis, high salt in trans to boost current). In this example, Current₁<Current₂<Current₃.

Devices 700, 700′, 700″ were prepared with varying liquids on the first (cis) and second (trans), and in some circumstances omitting the MspA nanopore. Unbalanced salt conditions across semipermeable membrane 701 caused solvent (water) to move through the membrane to balance the concentrations of salt in the cis and trans liquids. Circuitry 780 was used to measure the devices' capacitance, which for devices omitting the nanopore is proportional to the dielectric constant (ϵ), area (A), and thickness (d) of the membrane. It was assumed that the dielectric constant and thickness of membrane 701 substantially do not change due to osmosis, and that therefore any change in measured capacitance was substantially due to changes in the membrane's area, e.g., because the volume of solvent on the trans side changes over time.

FIGS. 8A-8C illustrate plots of the measured normalized capacitance as a function of time for the membrane described with reference to FIG. 7B. More specifically, plots 801, 802, and 803 respectively illustrated in FIGS. 8A, 8B, and 8C include the normalized capacitance as a function of time for device 700′ in which the trans side included twice the KCl concentration of the cis side and the nanopore was omitted. Trace 811 illustrated in FIG. 8A illustrates the normalized capacitance for an example in which membrane 701 included the low molecular weight diblock copolymer (LMW) and in which the cis liquid included 100 mM KCl and the trans liquid included 200 mM KCl. Trace 821 illustrated in FIG. 8A illustrates the normalized capacitance for an example in which membrane 701 included the low molecular weight diblock copolymer (LMW) and in which the cis liquid included 500 mM KCl and the trans liquid included 1000 mM KCl. Traces 811 and 821 both may be seen in FIG. 8A to start at a normalized capacitance of 1.0 at a time of zero minutes, and to increase over the approximately 25 minute measurement period to normalized capacitances, respectively, of about 1.7 and about 1.8. From FIG. 8A, it may be understood that for both devices, the difference in salt concentrations across the membrane caused increases in capacitance over about 20-25 minutes. These increases in capacitance were attributed to changes in the membrane's area that were caused by osmosis driven by the difference in salt concentrations. Differences in the absolute amount of salt in the cis and trans liquids, in the absence of a compound to balance the resulting osmotic pressure, were observed to result in different capacitances over time, attributed to different changes in the membrane's area caused by different amounts of osmosis during the measurement period.

Trace 812 illustrated in FIG. 8B illustrates the normalized capacitance for an example in which membrane 701 included the high molecular weight diblock copolymer (HMW) and in which the cis liquid included 100 mM KCl and the trans liquid included 200 mM KCl. Trace 822 illustrated in FIG. 8B illustrates the normalized capacitance for an example in which membrane 701 included the high molecular weight diblock copolymer and in which the cis liquid included 150 mM KCl and the trans liquid included 300 mM KCl. Trace 832 illustrated in FIG. 8B illustrates the normalized capacitance for an example in which membrane 701 included the high molecular weight diblock copolymer and in which the cis liquid included 500 mM KCl and the trans liquid included 1000 mM KCl. Traces 812, 822, and 832 may be seen in FIG. 8B to start at a normalized capacitance of 1.0 at a time of zero minutes, and to increase over about a 15 minute measurement period to normalized capacitances, respectively, of about 1.1, about 1.4, and about 1.8. From FIG. 8B, it may be understood that for each of the devices, the difference in salt concentrations across the membrane caused an increase in capacitance. These increases in capacitance were attributed to changes in the membrane's area that were caused by osmosis driven by the difference in salt concentrations, even when the absolute amount of salt in the cis and trans liquids differed. Differences in the absolute amount of salt in the cis and trans liquids, in the absence of a compound to balance the resulting osmotic pressure, were observed to result in different capacitances, attributed to different changes in the membrane's area caused by different amounts of osmosis during the measurement period.

Trace 813 illustrated in FIG. 8C illustrates the normalized capacitance for an example in which membrane 701 included DPhPC and in which the cis liquid included 100 mM KCl and the trans liquid included 200 mM KCl. Trace 823 illustrated in FIG. 8B illustrates the normalized capacitance for an example in which membrane 701 included the low molecular weight diblock copolymer (LMW) and in which the cis liquid included 100 mM KCl and the trans liquid included 200 mM KCl. Trace 833 illustrated in FIG. 8C illustrates the normalized capacitance for an example in which membrane 701 included the high molecular weight diblock copolymer (HMW) and in which the cis liquid included 100 mM KCl and the trans liquid included 200 mM KCl. Traces 813, 823, and 833 may be seen in FIG. 8C to start at a normalized capacitance of 1.0 at a time of zero minutes, and to increase over an approximately minute measurement period to normalized capacitances, respectively, of about 1.8, about 1.7, and about and about 1.2. From FIG. 8C, it may be understood that for each of the devices, the difference in salt concentrations across the membrane caused an increase in capacitance. These increases in capacitance were attributed to changes in the membrane's area that were caused by osmosis driven by the difference in salt concentrations, in the absence of a compound to balance the resulting osmotic pressure, even when the type of membrane differed. Differences in the membrane's composition were observed to result in different capacitances, attributed to different changes in the membrane's area caused by different amounts of osmosis during the measurement period.

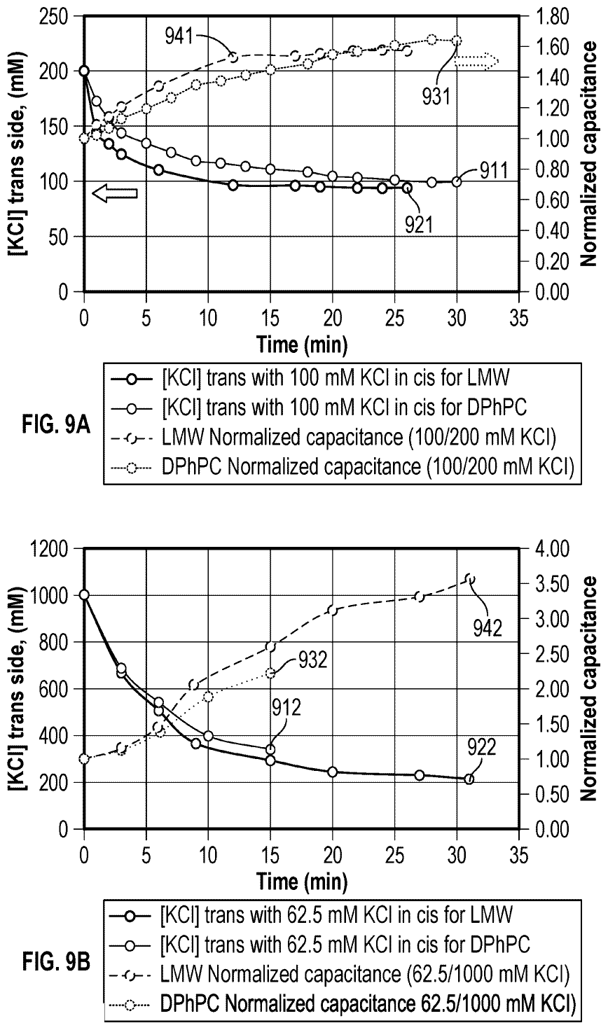

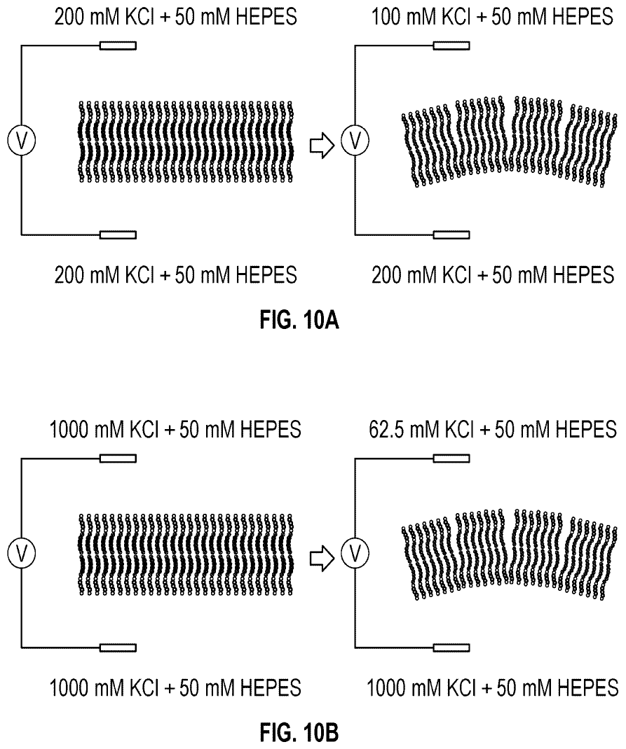

FIGS. 9A-9B illustrate plots of the measured salt concentration and normalized capacitance for the membrane described with reference to FIG. 7B. FIGS. 10A-10B schematically illustrate changes to the membrane described with reference to FIG. 7B during the measurements described with reference to FIGS. 9A-9B. Referring first to FIG. 9A, trace 911 illustrates the trans side concentration of KCl for an example in which membrane 701 included the low molecular weight diblock amphiphilic copolymer (LMW) and the initial cis and trans side concentration of KCl was 200 mM+50 mM HEPES. At time=0, the cis side concentration was changed to 100 mM KCl+50 mM HEPES, following which the trans side concentration of KCl changed due to osmosis across the membrane. Trace 921 illustrates the trans side concentration of KCl for an example in which membrane 701 included DPhPC and the initial cis and side concentration of KCl was 200 mM+50 mM HEPES. At time=0, the cis side concentration was changed to 100 mM KCl+50 mM HEPES, following which the trans side concentration of KCl changed due to osmosis across the membrane. Traces 911 and 921 may be seen in FIG. 9A to both begin at 200 mM, and to decrease over about a 25 minute period to about 100 mM for trace 921, or over about a 30 minute period to about 100 mM for trace 911, corresponding to osmotic equilibration of the KCl concentration across the membrane. Trace 931 illustrates the normalized capacitance for the same example as for trace 911, and trace 941 illustrates the normalized capacitance for the same example as for trace 921. Traces 931 and 941 may be seen in FIG. 9A to both begin at 1.0, and to increase over about a 25 minute period to about 1.5 for trace 941, or over about a 30 minute period to about 1.6 for trace 931, corresponding to deformation caused by osmotic equilibration of the KCl concentration across the membrane, e.g., such as illustrated in FIG. 10A. From FIG. 9A, it may be understood that a relatively low difference in salt concentration (about a factor of 2) between the trans and cis sides may equilibrate over time, in the absence of a compound to balance the resulting osmotic pressure, causing an increase in capacitance.

Referring now to FIG. 9B, trace 912 illustrates the trans side concentration of KCl for an example in which membrane 701 included the low molecular weight diblock amphiphilic copolymer and the initial cis and trans side concentration of KCl was 1000 mM+50 mM HEPES. At time=0, the cis side concentration was changed to 62.5 mM KCl+50 mM HEPES, following which the trans side concentration of KCl changed due to osmosis across the membrane. Trace 922 illustrates the trans side concentration of KCl for an example in which membrane 701 included DPhPC and the initial cis and trans side concentration of KCl was 1000 mM+50 mM HEPES. At time=0, the cis side concentration was changed to 62.5 mM KCl+50 mM HEPES, following which the trans side concentration of KCl changed due to osmosis across the membrane. Traces 912 and 922 may be seen in FIG. 9B to both begin at 1000 mM, and to decrease over about a 15 minute period to about 350 mM for trace 912, or over about a 32 minute period to about 200 mM for trace 922, corresponding to the device tending towards, but not yet reaching, osmotic equilibration of the KCl concentration across the membrane. Trace 932 illustrates the normalized capacitance for the same example as for trace 912, and trace 942 illustrates the normalized capacitance for the same example as for trace 922. Traces 932 and 942 may be seen in FIG. 9B to both begin at 1.0, and to increase over about a 15 minute period to about 2.3 for trace 932, or over about a 32 minute period to about 3.5 for trace 931, corresponding to deformation caused by osmotic equilibration of the KCl concentration across the membrane, e.g., such as illustrated in FIG. 10B. From FIG. 9B, it may be understood that a relatively high difference in salt concentration (about a factor of 16) between the trans and cis sides may change over time, in the absence of a compound to balance the resulting osmotic pressure, causing an increase in capacitance, and that still further changes may occur on longer timescales.

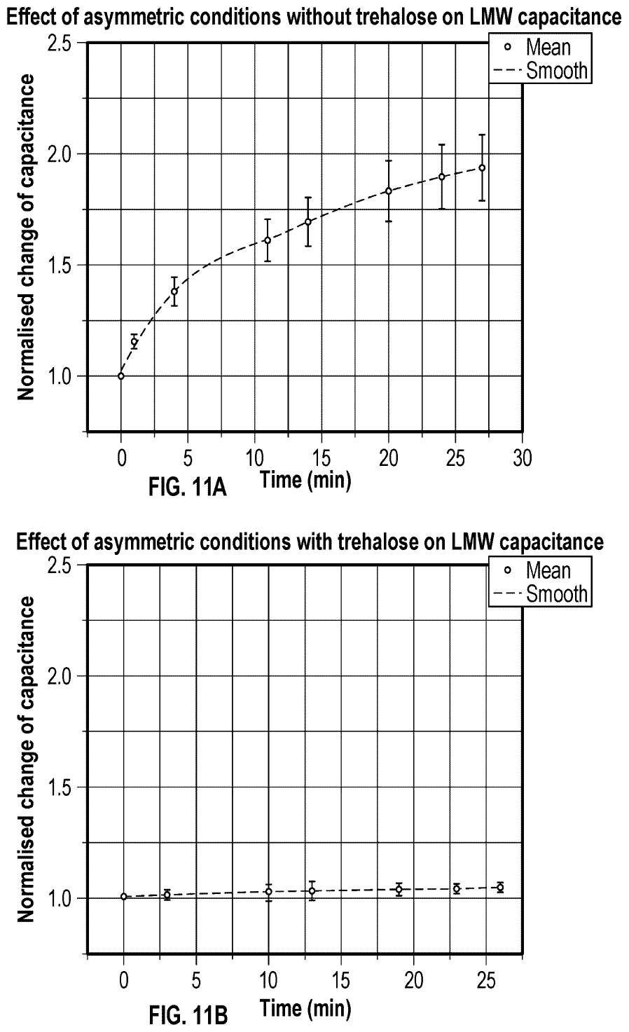

FIGS. 11A-11B illustrate plots of the normalized capacitance as a function of time for the membranes described with reference to FIGS. 7B and 7C. In the examples shown in FIG. 11A and 11B, the membrane was the low molecular weight amphiphilic diblock copolymer (LMW), and the initial concentration of KCl was 1000 mM on both the cis and trans side. In FIG. 11A, at time=0, the asymmetric condition of 250 mM KCl on the cis side and 1000 mM on the trans side was generated. It may be seen in FIG. 11A that the normalized capacitance increased from 1.0 at time=0, to about 1.9 at around 27 minutes. In comparison, in FIG. 11B, at time=0, the asymmetric condition of 250 mM KCl plus 500 mM trehalose (example compound to balance osmotic pressure caused by salt asymmetry) on the cis side and 1000 mM on the trans side was generated. It may be seen in FIG. 11B that the normalized capacitance was relatively stable, increasing from 1.0 at time=0, to less than about 1.1 at around 27 minutes. From FIGS. 11A-11B, it may be understood that the addition of trehalose to the asymmetric condition resulted in a significantly lower increase in normalized capacitance (FIG. 11B) than was observed from the asymmetric condition without trehalose (FIG. 11A). This lower increased in normalized capacitance was attributed to the trehalose on the cis side substantially balancing the osmotic pressure caused by the difference in salt concentrations between the cis and trans sides, and thus substantially inhibiting the deformation of the membrane such as described with reference to FIGS. 8A-8C, 9A-9B, and 10A-10B.

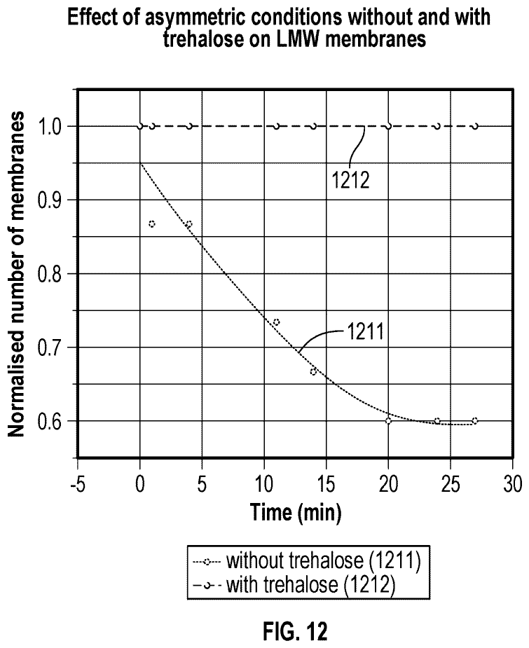

FIG. 12 illustrates a plot of the measured normalized number of membranes as a function of time for the membranes described with reference to FIGS. 7B and 7C. Trace 1211 was generated by preparing an array of 16 low molecular weight amphiphilic diblock copolymer membranes and applying the asymmetric condition described with reference to FIG. 11A at time=0, and measuring the normalized number of membranes as a function of time. Trace 1212 was generated by preparing an array of 16 low molecular weight amphiphilic diblock copolymer (LMW) membranes and applying the asymmetric condition described with reference to FIG. 11B at time=0, and measuring the normalized number of membranes as a function of time. Membrane formation was tracked by electrical current. When a membrane is formed, it makes an insulating layer that causes the current to drop to zero. Accordingly, a current drop was correlated to membrane formation. To check that the current drop was caused by membrane formation rather than a clog, a 1V pulse was applied for one second. A recovery of the current was correlated to loss of a membrane (loss of the insulating layer), meaning that a membrane had been formed. If the current instead remained at zero, it was interpreted as a clog. From FIG. 12, it may be seen that the normalized number of membranes for trace 1211 decreased from about 0.95 at time=0 to about 0.6 at around 27 minutes, while the normalized number of membranes for trace 1212 remained approximately at 1.0 for the 27 minutes of the measurement. From FIG. 12, it may be understood that the addition of trehalose to the asymmetric condition (trace 1212) significantly stabilized the membranes whereas the absence of trehalose resulting in relatively unstable membranes.

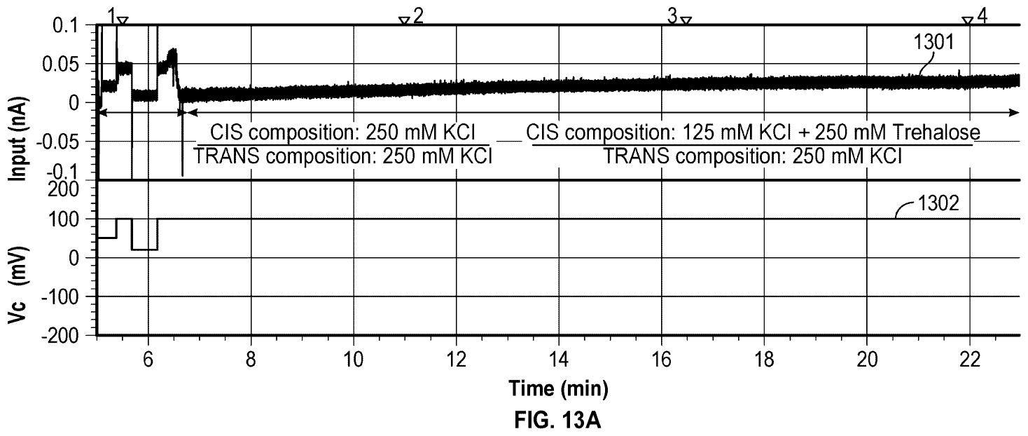

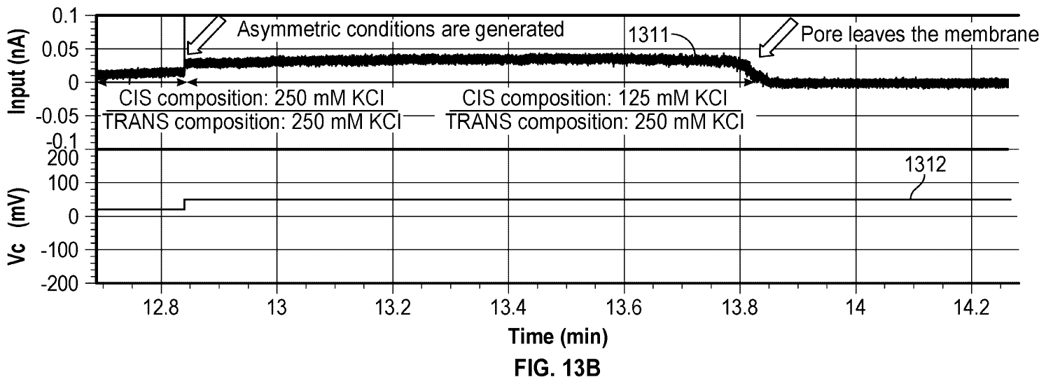

FIGS. 13A-13B illustrate plots of measured current and voltage as a function of time for the devices described with reference to FIGS. 7C and 7B. More specifically, the plots illustrated in FIG. 13A were prepared using the device illustrated in FIG. 7C, including MspA nanopore 710 in the low molecular weight amphiphilic diblock copolymer, and an initial concentration of 250 mM KCl in the aqueous compositions on both the cis and trans sides. Starting at a time of about 7 minutes, an asymmetrical condition in which the composition of the cis fluid was changed to 125 mM KCl and 250 mM trehalose. It may be seen in FIG. 13A that the current 1301 through the nanopore generally follows the applied voltage 1302, and increases over time from about 0.1 nA at the time the asymmetrical condition is applied, to about 0.3 nA at around 23 minutes. The plots illustrated in FIG. 13B were prepared using the device illustrated in FIG. 7B, including MspA nanopore 710 in the low molecular weight amphiphilic diblock copolymer, and an initial concentration of 250 mM KCl on both the cis and trans sides. Starting at a time of about 12.82 minutes, an asymmetrical condition in which the composition of the cis fluid was changed to 125 mM KCl (without trehalose). It may be seen in FIG. 13B that the current 1311 through the nanopore generally follows the applied voltage 1312 at a level of about 0.04 nA for only about one minute before decreasing to a value of 0.0 nA at a time of about 13.8 minutes. The decrease in current to about 0.0 nA in trace 1311 was attributed to the nanopore leaving the membrane due to deformation of the membrane caused by the osmotic pressure imbalance arising from the asymmetrical salt condition. In comparison, the relatively stable current in trace 1301 in FIG. 13A was attributed to the trehalose stabilizing the membrane by offsetting the osmotic pressure imbalance that otherwise would arise from the asymmetrical salt condition, thus allowing the nanopore to pass current normally over the course of time.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A device, comprising:
a fluidic well comprising a barrier, the barrier comprising a first side and a second side;
a first fluid within the fluidic well and in contact with the first side of the barrier, the first fluid having a first composition comprising a first concentration of a salt; and
a second fluid within the fluidic well and in contact with the second side of the barrier, the second fluid having a second composition comprising a second concentration of the salt that is different than the first concentration of the salt, a difference between the first and second concentrations of the salt generating a first osmotic pressure across the barrier,
the second composition further comprising a concentration of a compound other than the salt, the concentration of the compound generating a second osmotic pressure across the barrier that opposes and substantially balances the first osmotic pressure.

2. The device of claim 1, wherein the first concentration of the salt is between about 1.1 and about 50 times the second concentration of the salt.

3-4. (canceled)

5. The device of claim 1, wherein the first concentration of the salt is above about 150 mM, and wherein the second concentration of the salt is below about 100 mM.

6. (canceled)

7. The device of claim 1, wherein the first composition substantially does not include the compound.

8. The device of claim 1, wherein the concentration of the compound is between about 1.1 and about 50 times the first concentration of the salt.

9-10. (canceled)

11. The device of claim 1, wherein the concentration of the compound is above about 100 mM.

12. The device of claim 1, wherein the compound is charge neutral.

13. The device of claim 1, wherein the compound increases viscosity of the second fluid.

14. The device of claim 1, wherein the compound comprises an alcohol, a protein, or a polysaccharide.

15-16. (canceled)

17. The device of claim 14, wherein the polysaccharide comprises trehalose or a cyclodextrin.

18-20. (canceled)

21. The device of claim 1, wherein:
the first composition comprises a first concentration of an aqueous buffer,
the second composition comprises a second concentration of the aqueous buffer, and
the first concentration of the aqueous buffer is approximately equal to the second concentration of the aqueous buffer.

22. The device of claim 1, further comprising a nanopore disposed within the barrier and providing an aperture fluidically coupling the first side to the second side,
wherein a portion of the salt moves from the second side of the barrier to the first side of the barrier through the aperture.

23. (canceled)

24. The device of claim 22, wherein the compound substantially does not move from the second side of the barrier to the first side of the barrier through the aperture.

25. The device of claim 22, wherein the compound is larger in at least one dimension than the aperture.

26-29. (canceled)

30. The device of claim 29, further comprising:
a polymerase in the second composition or coupled to the nanopore or the barrier; and
first and second polynucleotides,
wherein the second composition further comprises a plurality of nucleotides, and the polymerase is for sequentially adding nucleotides of the plurality to the first polynucleotide using a sequence of the second polynucleotide.

31. The device of claim 30, further comprising:
a first electrode configured to contact the first fluid;
a second electrode configured to contact the second fluid; and
circuitry in operable communication with the first and second electrodes and configured to detect changes in an electrical characteristic of the aperture responsive to the polymerase sequentially adding nucleotides of the plurality to the first polynucleotide using a sequence of the second polynucleotide.

32. A sequencing method, comprising:
using the circuitry of the device of claim 31 to detect changes in the electrical characteristic of the aperture responsive to the polymerase sequentially adding nucleotides of the plurality to the first polynucleotide using a sequence of the second polynucleotide.

33. (canceled)

34. A device, comprising:
a fluidic well comprising a barrier, the barrier comprising a first side and a second side;
a first fluidic reservoir having a first amount of a salt therein;
a second fluidic reservoir having a second amount of the salt, and an amount of a compound, therein;
at least one fluidic channel for:
receiving a first solvent in the first fluidic reservoir such that the first solvent dissolves the first amount of the salt to form a first composition;
contacting the first side of the barrier with the first composition;
receiving a second solvent in the second fluidic reservoir such that the second solvent dissolves the second amount of the salt and the amount of the compound to form a second composition; and
contacting the second side of the barrier with the second composition,
wherein the second concentration of the salt in the second composition is different than the first concentration of the salt in the first composition so as to generate a first osmotic pressure across the barrier, and
wherein the concentration of the compound in the second composition generates a second osmotic pressure across the barrier that opposes and substantially balances the first osmotic pressure.

35. (canceled)

36. The device of claim 34, further comprising:
the first and second solvents;
the first and second compositions; and
a nanopore disposed within the barrier and providing an aperture fluidically coupling the first side to the second side,
wherein a portion of the salt in the second composition moves from the second side of the barrier to the first side of the barrier through the aperture.

37-39. (canceled)

40. The device of claim 36, wherein the compound in the second composition substantially does not move from the second side of the barrier to the first side of the barrier through the aperture.

41-43. (canceled)

44. The device of claim 36, further comprising:
a polymerase in the second composition or coupled to the nanopore or the barrier; and
first and second polynucleotides.

45. The device of claim 44, wherein the second composition further comprises a plurality of nucleotides, and wherein the polymerase is for sequentially adding nucleotides of the plurality to the first polynucleotide using a sequence of the second polynucleotide.

46. The device of claim 45, further comprising:
a first electrode configured to contact the first composition;
a second electrode configured to contact the second composition; and
circuitry in operable communication with the first and second electrodes and configured to detect changes in an electrical characteristic of the aperture that are responsive to the polymerase sequentially adding nucleotides of the plurality to the first polynucleotide using a sequence of the second polynucleotide.

47. A sequencing method, comprising:
using the circuitry of the device of claim 46 to detect changes in the electrical characteristic of the aperture that are responsive to the polymerase sequentially adding nucleotides of the plurality to the first polynucleotide using a sequence of the second polynucleotide.

48. A method of osmotically balancing a barrier, the method comprising:
contacting a first side of a barrier with first composition comprising a first concentration of a salt;
contacting a second side of the barrier with a second composition comprising (i) a second concentration of the salt, and (ii) a concentration of a compound other than the salt;
generating a first osmotic pressure across the barrier using a difference between the first and second concentrations of the salt; and
generating a second osmotic pressure across the barrier using the concentration of the compound, the second osmotic pressure opposing and substantially balancing the first osmotic pressure.

49. (canceled)
